(19) United States
(12) Patent Application Publication
(10) Pub. No.: US 2016/0302657 A1
(43) Pub. Date: Oct. 20, 2016

(54) DUAL CHANNELED GYNECOLOGIC SPECULUM WITH SPECIMEN COLLECTION AND DIAGNOSTIC IMAGING CAPABILITIES

(71) Applicants: Jacqueline Marie Hussey, Winfield, IL (US); Kevin Ignatius Hussey, Winfield, IL (US); Scot Herbst, Santa Cruz, CA (US)

(72) Inventors: Jacqueline Marie Hussey, Winfield, IL (US); Kevin Ignatius Hussey, Winfield, IL (US); Scot Herbst, Santa Cruz, CA (US)

(21) Appl. No.: 15/099,972

(22) Filed: Apr. 15, 2016

Related U.S. Application Data
(60) Provisional application No. 62/147,698, filed on Apr. 15, 2015.

(51) Int. Cl. A61B 1/32; A61B 1/00; A61B 17/42; A61B 1/04; A61B 1/06

(52) U.S. Cl. CPC A61B 1/32; A61B 1/04; A61B 1/06; A61B 17/42; A61B 1/00154

(57) ABSTRACT

The present invention generally relates a medical tool for investigating bodily orifices. Specifically, this invention relates to a dual chambered speculum for the performance of medical examinations and procedures. Furthermore, the invention relates a method of testing to be used in conjunction with current protocols.

### CROSS REFERENCE To RELATED APPLICATIONS

This application claims the benefit of U.S. Provisional Patent Application No. 62/147,698 Filed Apr. 15, 2015, the entire disclosure of which is incorporated herein by reference.

### FIELD OF INVENTION

Embodiments of the present invention are generally directed towards an apparatus for performing gynecological and other diagnostic procedures. Specifically, embodiments of the present invention are directed towards a dual-chambered speculum that provides a light source and an imaging means for taking and collecting images for cytological, microbiological and pathological analysis of in natura specimen.

### BACKGROUND

Conventional speculums are prone to discomfort and pain since they are blindly inserted into a patient's orifice, and the practitioner, based on feel and experience, attempts to position the speculum in appropriate setting. In doing so, the speculum may be inserted more deeply within the patient's orifice than necessary or desirable, creating pain and unpleasantness. This may lead to an automatic flexing of internal muscles such that the orifice is involuntarily tightened, in which case the conventional speculum forcibly opens the orifice leading to more unease and irritation.

Further, conventional speculums are associated with manual examinations, and current protocol dictates any results of an examination are memorialized as practitioner's visual observations and any accompanying diagnostic test. As an example, results of a Pap Smear examination consist of the practitioner's visual observations and any microbiological, cytological and/or pathological tests. The drawback of this procedure is that it is prone to error. For example, a diagnostic test may reveal a possible malady, but a negative visual observation may lead a practitioner to conclude the test may have been false. On the other hand, the practitioner may observe something peculiar, but may conclude that it is insignificant if a diagnostic test reveals a false negative.

Therefore, there is a need in the art for a dual-chambered speculum that provides a light source and an imaging means for taking and collecting images for cytological, microbiological and pathological analysis of in natura specimen. These and other features and advantages of the present invention will be explained and will become obvious to one skilled in the art through review of the present application.

### SUMMARY OF THE INVENTION

Accordingly, there is a need in the art for a dual-chambered speculum that provides a light source and an imaging means for taking and collecting images for cytological, microbiological and pathological analysis of in natura specimen.

According to an embodiment of the present invention, a dual chambered speculum comprises: a speculum housing comprising a barrel portion, wherein said barrel portion is an elongated structure and comprises a first chamber and a second chamber, formed in the interior of said barrel portion of said speculum housing; said first chamber passing through substantially the entire length of the elongated structure barrel portion and open on both a first tool insertion end and a second end of said elongated structure opposite to the first tool insertion end, wherein said second end is a insertional terminal end of said dual chambered speculum; said second chamber being closed on a same end as said second end of said elongated structure opposite the first tool insertion end and configured to receive one or more of a light source and an imaging device.

According to an embodiment of the present invention, the speculum housing further comprises a handle portion, wherein said handle portion is connected to said barrel portion on at least one edge of said barrel portion.

According to an embodiment of the present invention, the handle portion is connected to said barrel portion on said at least one edge such that said barrel portion and said handle portion are angled approximately 20° to 150° from one another.

According to an embodiment of the present invention, the handle portion comprises an elongated body with a cavity formed therein.

According to an embodiment of the present invention, the cavity is configured to receive a body of one or more of said imaging device and said light source.

According to an embodiment of the present invention, a passage is formed between said cavity of said handle portion and said second chamber of said barrel portion.

According to an embodiment of the present invention, the passage formed between said cavity of said handle portion and said second chamber permits the passage of a flagella-like protrusion from one or more of said light source and imaging device to pass from said cavity of said handle portion into said second chamber.

According to an embodiment of the present invention, the cavity is further configured to retain a body of one or more of said light source and said imaging device therein.

According to an embodiment of the present invention, the first chamber is a substantially straight and tubular cavity formed entirely within said barrel portion.

According to an embodiment of the present invention, the first end of said barrel portion forms a flared tool insertion point with beveled ends.

According to an embodiment of the present invention, the flared tool insertion point leans approximately 45° and in such a manner that the tool insertion point extends for approximately 1.5-2 cm from the first end.

According to an embodiment of the present invention, the first chamber and second chamber are separated by a wall running the length of the elongated body between the first chamber and the second chamber.

According to an embodiment of the present invention, the second chamber is sealed on the same end as the second end of the first chamber.

According to an embodiment of the present invention, a dual chambered speculum, comprises: a speculum housing comprising a barrel portion, wherein said barrel portion comprises a first chamber and a second chamber, formed in the interior of said barrel portion of said speculum housing; said first chamber passing through substantially the entire length of the barrel portion and open on both a first tool insertion end and a second end of said barrel portion opposite to the first tool insertion end, wherein said second end is a insertional terminal end of said dual chambered speculum; said second chamber being closed on a same end as said second end of said elongated structure opposite the first tool insertion end and configured to receive one or more of a light source and an imaging device.

The foregoing summary of the present invention with the preferred embodiments should not be construed to limit the scope of the invention. It should be understood and obvious to one skilled in the art that the embodiments of the invention thus described may be further modified without departing from the spirit and scope of the invention.

### BRIEF DESCRIPTION OF THE DRAWINGS

FIG. 1 is a perspective view of a dual-chambered speculum in accordance with an embodiment of the present invention;

FIG. 2 is a perspective view of a dual-chambered speculum in accordance with an embodiment of the present invention;

FIG. 3 is a side view of a dual-chambered speculum adapted to receive a camera and a cytology brush in accordance with an embodiment of the present invention;

FIG. 4 is a side view of a dual-chambered speculum adapted to receive a camera and a cytology brush in accordance with an embodiment of the present invention;

FIG. 5 is a perspective view of a dual-chambered speculum supplemented with a camera and a cytology brush in accordance with an embodiment of the present invention;

FIG. 6 is a rear perspective view of a dual-chambered speculum supplemented with a camera and a cytology brush in accordance with an embodiment of the present invention;

FIG. 7 is a side perspective view of a dual-chambered speculum supplemented with a camera and a cytology brush in accordance with an embodiment of the present invention;

FIG. 8 is an opposite side perspective view of a dual-chambered speculum supplemented with a camera and a cytology brush in accordance with an embodiment of the present invention;

FIG. 9 is a rear perspective view of a dual-chambered speculum supplemented with a camera and a cytology brush in accordance with an embodiment of the present invention;

FIG. 10 is a side view of a dual-chambered speculum supplemented with a camera and a cytology brush in accordance with an embodiment of the present invention;

FIG. 11 is a side view of a dual-chambered speculum in accordance with an embodiment of the present invention;

FIG. 12 is a front perspective view of a dual-chambered speculum in accordance with an embodiment of the present invention;

FIGS. 13 is a side perspective view of a dual-chambered speculum in accordance with an embodiment of the present invention;

FIG. 14 is a side view of a dual-chambered in accordance with an embodiment of the present invention; and

FIG. 15 is a side perspective view of a dual-chambered speculum in accordance with an embodiment of the present invention.

### DETAILED SPECIFICATION

The present invention generally relates to a speculum. Specifically, embodiments of the present invention relate to a dual chambered speculum. In a preferred embodiment the dual chambered speculum has a first chamber and a second chamber. In the preferred embodiment, the first chamber may be used to house a light source, camera, imaging device, or any combination thereof (hereinafter the “imaging device”). In the preferred embodiment, the first chamber (hereinafter the “closed chamber” or “light chamber” or “imaging chamber”) is closed at the insertional terminal of the speculum. In the preferred embodiment, the second chamber (hereinafter the “tool chamber” or the “open chamber”) serves as a channel for guiding diagnostic tools for examination purposes and is thus opened at the insertional terminal. Certain embodiments of the present invention may include fewer components or additional components depending on the utilization and purpose for the dual chambered speculum.

According to an embodiment of the present invention, the body of the dual chambered speculum described herein is substantially pistol-shaped, with a handle portion that is substantially perpendicular to a barrel portion. In preferred embodiments, the handle portion and the barrel portion are at approximately 45° to 60° relative to each other. In alternative embodiments, the handle portion and the barrel portion are approximately 20° to 150° relative to each other. In the preferred embodiment, the handle portion is primarily formed from the end of the closed chamber that is opposite the insertional terminal of the speculum, while the barrel portion of the speculum is formed by both the open chamber and the closed chamber. In the preferred embodiment, the barrel portion of the speculum is a substantially tubular body with an interior cavity that is divided along the entirety of its length by a wall. In the preferred embodiment, this wall divides the open chamber from the closed chamber. In the preferred embodiment, the handle portion of the speculum connects to the end of the barrel portion that is opposite the insertional terminal of the speculum.

According to embodiments of the present invention, the barrel portion is intended to receive and/or house an imaging device therein. In a preferred embodiment, the imaging device is an assembly comprising imaging technology scaled to fit within an encasing cavity formed within the handle portion. As an example, the handle portion may be used to secure one or more of a fiber optic light source and digital camera within the encasing cavity. In such an example, a camera body of the digital camera is of sufficient thickness to be inserted within the handle portion. A flagella-like protrusion of the camera device containing the viewing lens extends from the handle portion to the barrel portion and is received in the closed chamber. Such flagella-like protrusion is of sufficient flexibility and thickness so that it may be curved and admitted into a lower, light chamber of the closed chamber for the purpose of visualizing the specimens and capturing images of such specimen. The opposite end of the imaging device may be connected to a monitor to provide real-time viewing of the internal anatomy and process the capture images. In certain embodiments, the flagella-like protrusion may incorporate the fiber optic light source as well. In other embodiments, the fiber optic light source may be separate from the camera body and flagella-like protrusion and be inserted into the same closed chamber or a separate section of the closed chamber configured to receive a fiber optic light source.

According to alternative embodiment, the handle portion is not configured to house the imaging device, but rather allows for a flagella-like protrusion to pass through the handle portion into the closed chamber. In still another embodiment, the handle portion is configured to allow passage of a connection means, such as fiber optic wiring, universal serial bus (USB) wiring, RJ-45 wiring, or any other type of wired connection means capable of transferring data and/or power to/from an imaging and/or light source contained in the closed chamber. One of ordinary skill in the art would appreciate that there are numerous types of wired connection means that could be used with embodiments of the present invention, and embodiments of the present invention are contemplated for use with any appropriate wired connection means. Similarly, in other embodiments, wired connection means may be exchanged for wireless connection means, such as devices configured for providing Bluetooth connections, Wi-Fi connections or devices configured to provide communications over any other wireless connections protocol.

According to an embodiment of the present invention, the closed chamber is closed at the insertional terminal located on the barrel portion of the speculum and open at the bottom of the handle portion of the speculum. In a preferred embodiment, the closed chamber encases portions of the light source, camera, or imaging source that are inserted into the closed chamber via the handle portion of the speculum. In the preferred embodiment, the closed chamber is a continuously formed cavity that bends at an angle where the barrel portion of the speculum meets the handle portion of the speculum. According to embodiments of the present invention, the closed chamber is intended to retain a light source and an imaging device. As an illustrative example, the closed chamber may be used to secure a fiber optic light source and digital camera. By having a wall block further advancement of the light source and imaging device, such source can be rested within the device without any unnecessarily prodding of the specimen.

According to an embodiment of the present invention, the open chamber is open at both ends. In a preferred embodiment, the open chamber is substantially straight and tubular cavity formed entirely within the barrel portion of the speculum. In the preferred embodiment, the open chamber is used to direct light and examination tools on the desired in natura specimen. As an illustrative example, the open chamber provides a direct channel for the utilization of diagnostic and examining instruments, such as a cytology brush.

According to an embodiment of the present invention, the open chamber further comprises a beveled end that is formed at the end of the barrel portion that is opposite the insertional terminal of the speculum, thereby forming an enlarged or flared tool insertion point. In a preferred embodiment, the beveled end leans approximately 45° in such a manner that the tool chamber extends approximately 1.5-2 cm further than the end of the light chamber.

According to embodiments of the present invention, the open chamber is situated on top of the closed chamber with the barrel portion of the speculum. With an imaging device being located on the bottom, a user of an embodiment of the present invention is able to examine a specimen without any inference from the protruding portions of the imaging device. Specifically, if the imaging device were situated on the upper level, the neck of the imaging device may block the entryway for a diagnostic tool, requiring the user to maneuver around the imaging device. With the imaging device situated on the bottom, access to the specimen is unimpeded by the tool itself. According to an alternate embodiment of the present invention, the dual chambers are situated adjacent to one another such that they share a common side wall.

According to embodiments of the present invention, the insertional terminal is also beveled. The beveled end leans approximately 45° in such a manner that the leading edge of the tool chamber extends approximately 1.5-2 cm further than the end of the light chamber at the insertional terminal. This provides a flared “lip” which can serve to provide leverage for retraction and/or manipulation of anatomical structures for enhanced visualization.

According to embodiments of the present invention, the dual chambered speculum is composed of polymer. In a preferred embodiment, the dual chambered speculum is composed of acrylic. In an alternative embodiment, the dual chambered speculum is composed of acrylic-like polymers. Other transparent and break resistant materials may be used in the assembly of the dual chambered speculum. One of ordinary skill in the art would appreciate that there are many suitable materials from which a speculum could be manufactured, and embodiments of the present invention are contemplated for use with any such material.

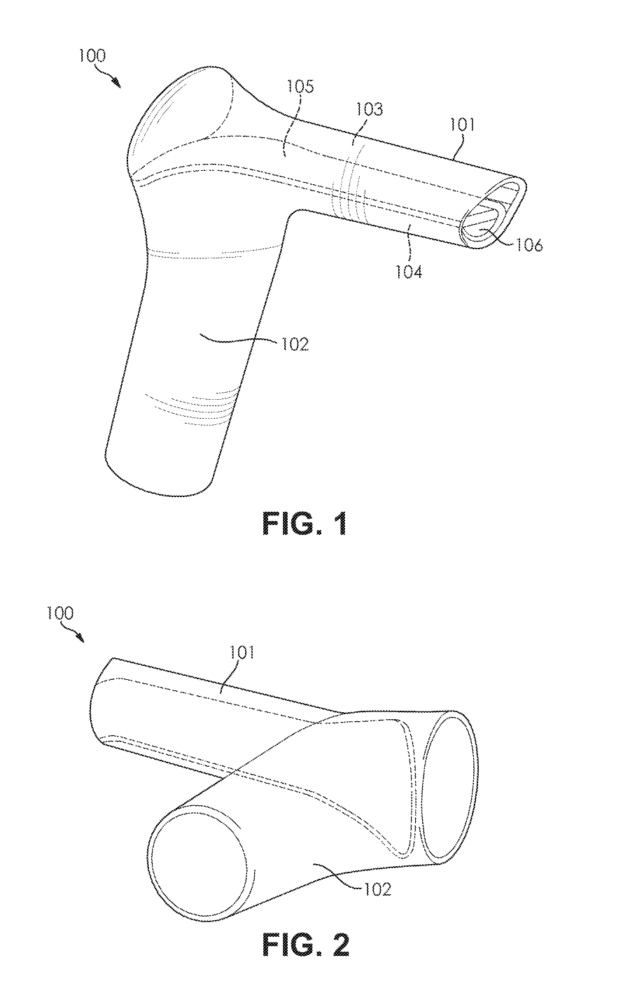

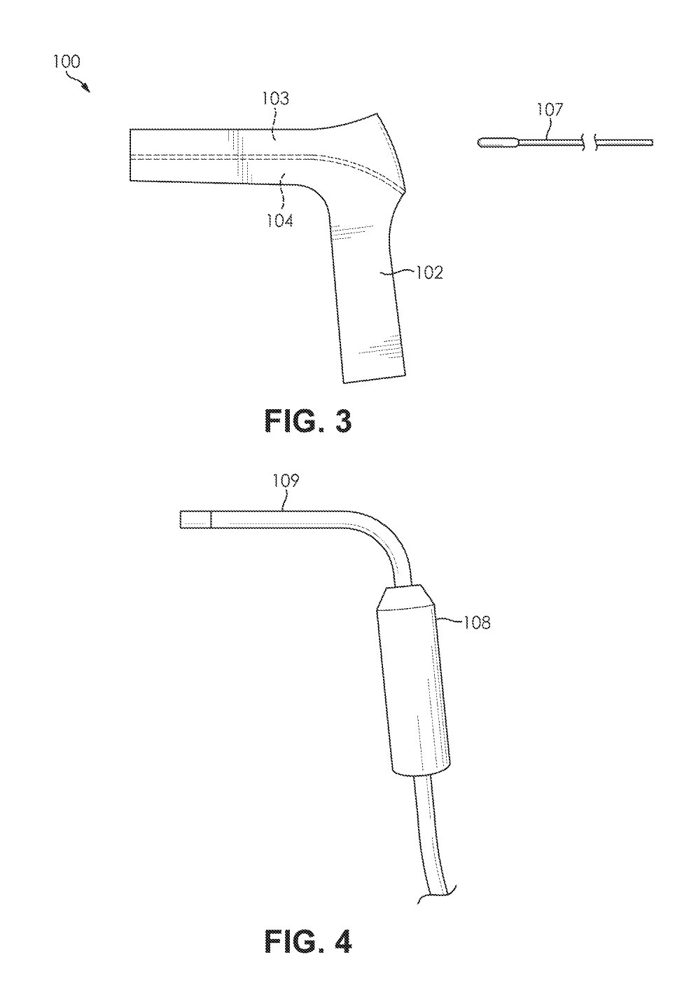

Turning to FIGS. 1-3, an exemplary embodiment of a dual chambered speculum is shown. In these figures, the dual chambered speculum 100 is comprised of a barrel portion 101 and handle portion 102. The barrel portion comprises a first chamber (i.e., open chamber) 103 and second chamber (i.e., closed chamber) 104. The first chamber 103 and second chamber 104 are separated by a wall 105 running the entire length of the barrel portion. The second chamber is a closed chamber, terminating at an end 106. An insertion tool 107 may be inserted into the first chamber 103 and pass through into the insertion terminal end of the barrel portion.

Turning to FIG. 4, an exemplary embodiment of an imaging device is shown. The imaging device comprises a imaging device body 108 and a flagella-like protrusion 109 comprising a camera and or light source.

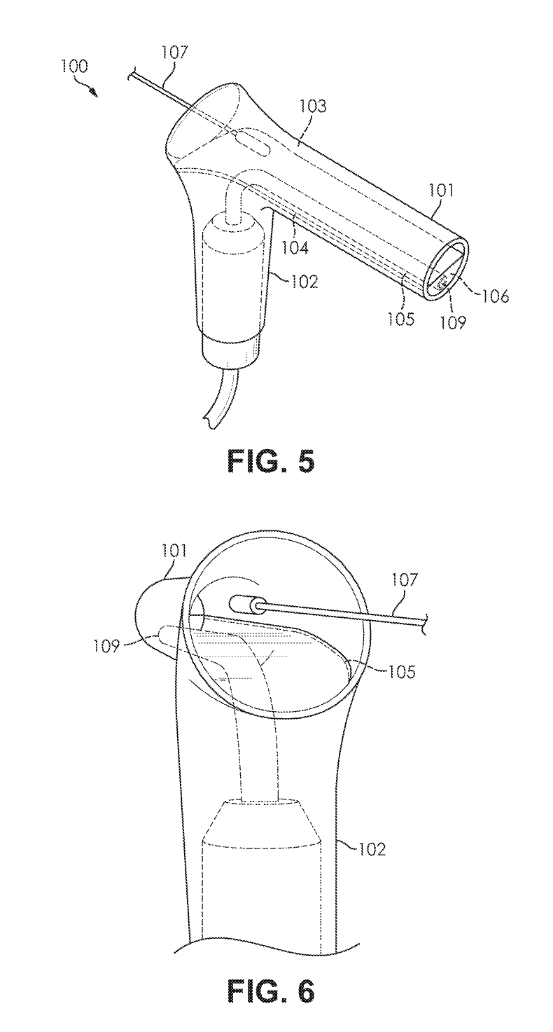

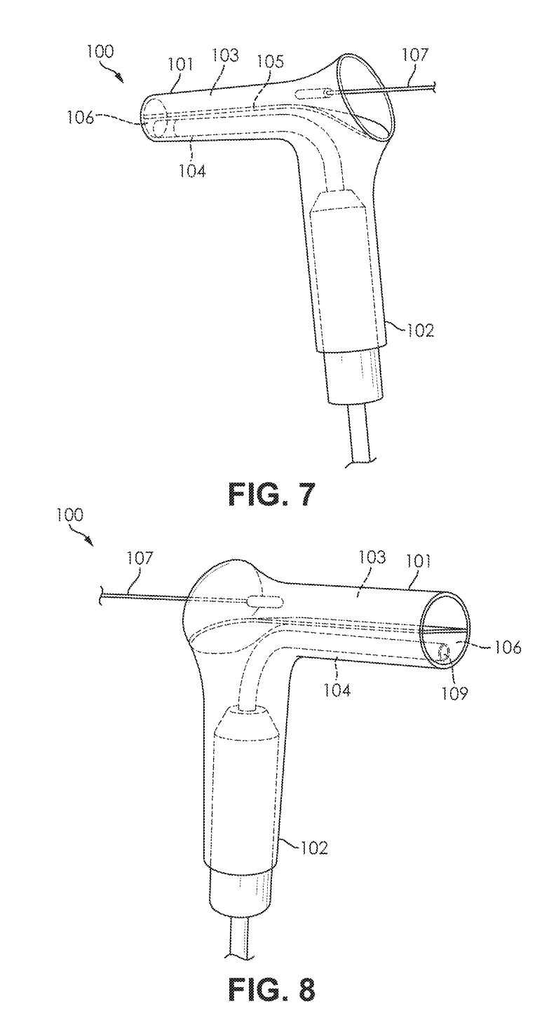

Turning to FIGS. 5-8, an exemplary embodiment of a dual chambered speculum is shown, in conjunction with an imaging device. In this figure, the imaging device body 108 is contained within a cavity within the handle portion of the dual chambered speculum 100. The flagella-like protrusion passes from the handle portion and through into the second chamber. An insertion tool 107 is shown entering the first chamber 103 of the barrel portion 101 of the dual chambered speculum 100.

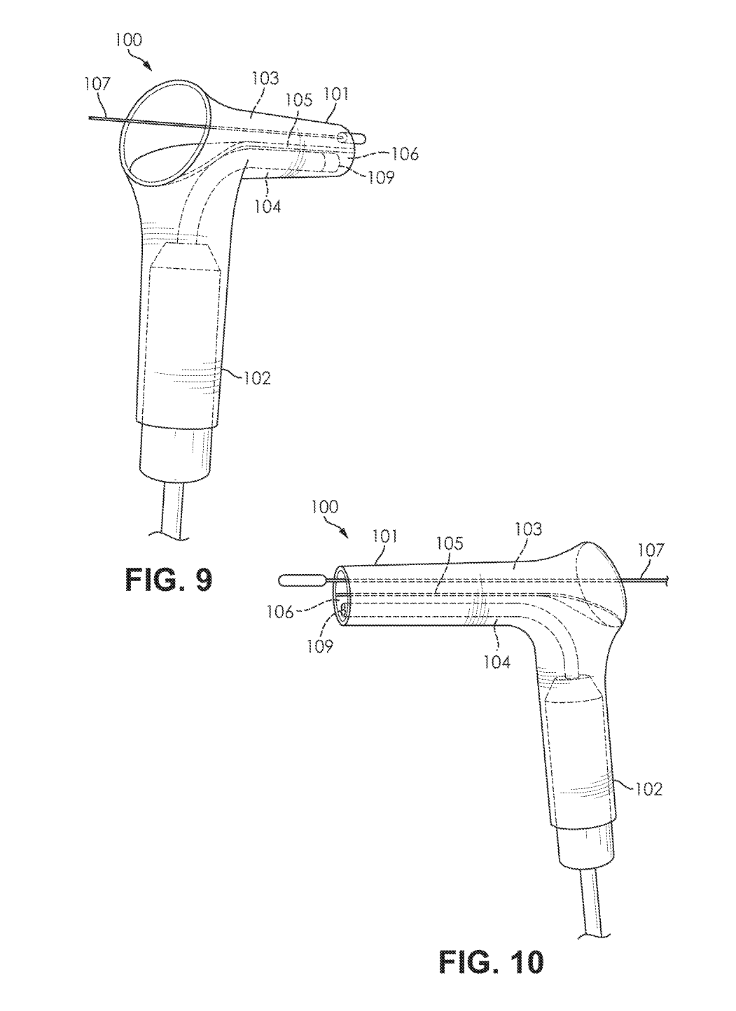

Turning to FIGS. 9-10, an exemplary embodiment of a dual chambered speculum is shown, in conjunction with an imaging device. In this figure, the imaging device body 108 is contained within a cavity within the handle portion of the dual chambered speculum 100. The flagella-like protrusion passes from the handle portion and through into the second chamber. An insertion tool 107 is shown passing through the first chamber 103 of the barrel portion 101 of the dual chambered speculum 100 and into and through the insertional terminal end.

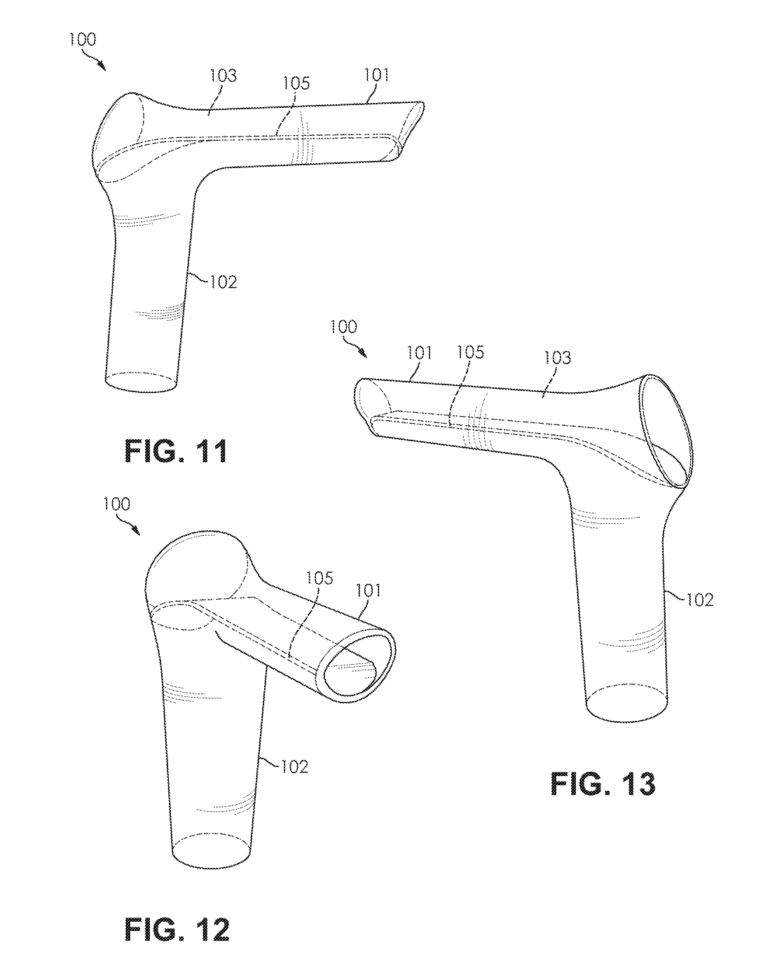

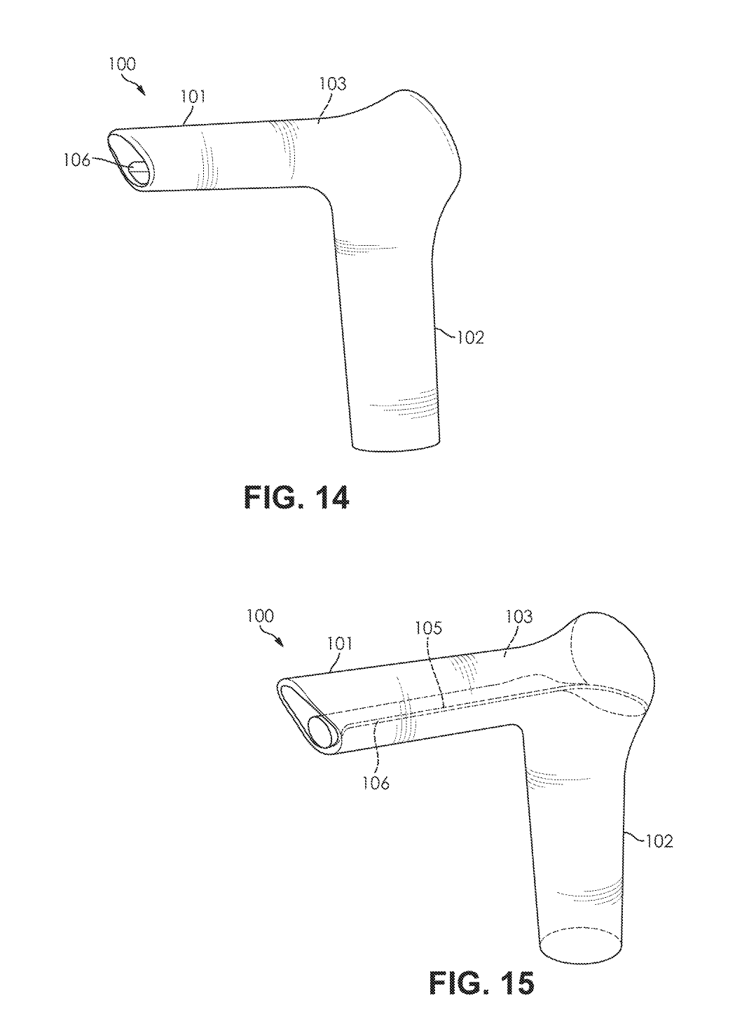

Turning to FIGS. 11-14, an exemplary embodiment of a dual chambered speculum is shown. In these figures, the dual chambered speculum 100 is comprised of a barrel portion 101 and handle portion 102. The barrel portion comprises a first chamber (i.e., open chamber) 103 and second chamber (i.e., closed chamber) 104. The first chamber 103 and second chamber 104 are separated by a wall 105 running the entire length of the barrel portion. The second chamber is a closed chamber, terminating at an end 106. An insertion tool 107 may be inserted into the first chamber 103 and pass through into the insertion terminal end of the barrel portion.

According to an embodiment of the present invention, the external form of the barrel portion is designed for a superior fit and performance over conventional speculum. First, the shape of the barrel portion is more consistent with the patient's normal cavity. Embodiments of the present invention are available in a variety of sizes such that a size that is best received by the patient's anatomy can be used for examination. By using the appropriate size the barrel portion to able to glide more easily through a patient's orifice without any unnecessary expansion of the orifice. In tandem with the shape, an embodiment of the present invention is more comfortable for a patient because it permits the practitioner to visualize the internal anatomy through use of the light and camera means. Since the practitioner can view the internal anatomy through a monitor, an embodiment of the present invention is inserted and guided with clear visualization. Embodiments of the present invention are inserted only as deeply as necessary and can be directed around the natural anatomy of the patient. Moreover, since the imaging device projects the internal anatomy, there is no unnecessary widening of the orifice that can cause pinching and/or pain. In addition, since viewing of the internal anatomy is done through a camera lens of the imaging device, such lens with zooming capabilities, can enable a clearer view of the in natura specimen without further and unnecessary prodding.

One advantage of embodiments of the present invention over conventional specula is the delivery of accuracy to the diagnostic procedures. Embodiments of the present invention permit the memorialization of the actual specimen which was observed by the practitioner through the imaging device. This enables further review of the specimen at a later time in light of any accompanying diagnostic test. Further, the memorialization permits the chronicling of the specimen for archive and record keeping. This will permit a review of a specimen over time and any morphological and physiological changes can be readily ascertained.

Accuracy pursuant to an embodiment of the present invention is also attributed to the light and imaging device. Most imaging devices, such as cameras, are accompanied with a multiple variety of lenses. Accordingly, embodiments of the present invention can provide different views of the specimen for a more comprehensive analysis.

Another benefit of an embodiment of the present invention is that it provides more flexibility in delivering care to a patient. If a qualified practitioner is not geographically available, projecting the visual examination remotely can permit a qualified practitioner to perform an immediate examination.

Embodiments of the present invention can be used for any number of medical procedures. For example, embodiments of the present invention can be used to perform gynecological exams such as Pap Smears and other general screening tests such as cervicography. Embodiments of the present invention may also be used for intrauterine device insertion, endometrial biopsies, and colposcopy. The various applications are not intended to be limited features of embodiments of the present invention.

As an illustrative example, embodiments of the present invention can be used for obstetric applications including not limited to evaluation for ruptured members and cervical change as well as investigating the etiology of bleeding during pregnancy.

It should be noted that the features illustrated in the drawings are not necessarily drawn to scale, and features of one embodiment may be employed with other embodiments as the skilled artisan would recognize, even if not explicitly stated herein. Descriptions of well-known components and processing techniques may be omitted so as to not unnecessarily obscure the embodiments.

According to embodiments of the present invention, a method for medical testing is disclosed herein. Current methods for testing include a two-step process in which a specimen is collected from a patient and undergoes cytological, microbiological and pathological testing followed by an in vitro screening. As an illustration, testing for cervical cancer includes obtaining cervical samples and assaying them for any abnormalities. Following such testing, the samples undergo a screening process, which may include a DNA analysis, such as a high risk HPV screen. The medical testing protocol may include an imaging step. Specifically, following the cytology assays and screening, the imaging step collects the visualization of the in natura specimen and documents such data for review at a later date. In other embodiments, the imaging step may occur at prior to one or both of the cytology step and the screening step. Accordingly, with this new method, there is a tri-tier testing of specimen that enables a more accurate diagnosis and monitoring of a potential malady.

It should be noted that the features illustrated in the drawings are not necessarily drawn to scale, and features of one embodiment may be employed with other embodiments as the skilled artisan would recognize, even if not explicitly stated herein. Descriptions of well-known components may be omitted so as to not unnecessarily obscure the embodiments.

While multiple embodiments are disclosed, still other embodiments of the present invention will become apparent to those skilled in the art from this detailed description. The invention is capable of myriad modifications in various obvious aspects, all without departing from the spirit and scope of the present invention. Accordingly, the drawings and descriptions are to be regarded as illustrative in nature and not restrictive.

1. A dual chambered speculum, said dual chambered speculum comprising:
a speculum housing comprising a barrel portion, wherein said barrel portion is an elongated structure and comprises a first chamber and a second chamber, formed in the interior of said barrel portion of said speculum housing;
said first chamber passing through substantially the entire length of the elongated structure barrel portion and open on both a first tool insertion end and a second end of said elongated structure opposite to the first tool insertion end, wherein said second end is a insertional terminal end of said dual chambered speculum;
said second chamber being closed on a same end as said second end of said elongated structure opposite the first tool insertion end and configured to receive one or more of a light source and an imaging device.

2. The dual chambered speculum of claim 1, wherein said speculum housing further comprises a handle portion, wherein said handle portion is connected to said barrel portion on at least one edge of said barrel portion.

3. The dual chambered speculum of claim 2, wherein said handle portion is connected to said barrel portion on said at least one edge such that said barrel portion and said handle portion are angled approximately 20° to 150° from one another.

4. The dual chambered speculum of claim 2, wherein said handle portion comprises an elongated body with a cavity formed therein.

5. The dual chambered speculum of claim 4, wherein said cavity is configured to receive a body of one or more of said imaging device and said light source.

6. The dual chambered speculum of claim 4, wherein a passage is formed between said cavity of said handle portion and said second chamber of said barrel portion.

7. The dual chambered speculum of claim 6, wherein said passage formed between said cavity of said handle portion and said second chamber permits the passage of a flagella-like protrusion from one or more of said light source and imaging device to pass from said cavity of said handle portion into said second chamber.

8. The dual chambered speculum of claim 7, wherein said cavity is further configured to retain a body of one or more of said light source and said imaging device therein.

9. The dual chambered speculum of claim 1, wherein said first chamber is a substantially straight and tubular cavity formed entirely within said barrel portion.

10. The dual chambered speculum of claim 1, wherein said first end of said barrel portion forms a flared tool insertion point with beveled ends.

11. The dual chambered speculum of claim 10, wherein said flared tool insertion point leans approximately 45° and in such a manner that the tool insertion point extends for approximately 1.5-2 cm from the first end.

12. The dual chambered speculum of claim 1, wherein said first chamber and second chamber are separated by a wall running the length of the elongated body between the first chamber and the second chamber.

13. The dual chambered speculum of claim 1, wherein said second chamber is sealed on the same end as the second end of the first chamber.

14. A dual chambered speculum, said dual chambered speculum comprising:
a speculum housing comprising a barrel portion, wherein said barrel portion comprises a first chamber and a second chamber, formed in the interior of said barrel portion of said speculum housing;
said first chamber passing through substantially the entire length of the barrel portion and open on both a first tool insertion end and a second end of said barrel portion opposite to the first tool insertion end, wherein said second end is a insertional terminal end of said dual chambered speculum;
said second chamber being closed on a same end as said second end of said elongated structure opposite the first tool insertion end and configured to receive one or more of a light source and an imaging device.
